(19) United States
(12) Patent Application Publication
(10) Pub. No.: US 2017/0299607 A1
(43) Pub. Date: Oct. 19, 2017

(54) Methods For Providing A Prognosis Of Pregnancy

(71) Applicant: Map IP Holding Limited, Ely (GB)

(72) Inventors: Raymond Kruse Iles, Ely (GB); Stephen Andrew Butler, Ely (GB)

(21) Appl. No.: 15/513,644

(22) PCT Filed: Sep. 24, 2015
(86) PCT No.: PCT/GB2015/052770

§ 371 Date: Mar. 23, 2017

(30) Foreign Application Priority Data
Sep. 24, 2014 (GB) 1416866.0
Jun. 11, 2015 (GB) 1510216.3

(51) Int. Cl. G01N 33/68

(52) U.S. Cl. CPC G01N 33/6851; G01N 33/689; G01N 2800/368

(57) ABSTRACT

The invention relates to a method of providing a prognosis for a pregnancy comprising subjecting a sample to direct mass spectral analysis, and comparing the spectra resulting from said analysis to spectra generated from samples obtained from 5 normal pregnancies. The invention describes subjecting a sample of maternal bodily fluid and/or fluids in which an embryo is cultured, or kept in, to direct matrix assisted laser desorption time of flight mass spectrometry and detection of spectral masses between 2,000 and 100,000 m/z. The changes in spectral masses, leading to changes in the profile of the spectra, are characteristic of disorders of fetal development and 10 pregnancy.

The invention relates to a method of providing a prognosis for a pregnancy comprising subjecting a sample to direct mass spectral analysis, and comparing the spectra resulting from said analysis to spectra generated from samples obtained from normal pregnancies. The invention describes subjecting a sample of maternal bodily fluid and/or fluids in which an embryo is cultured, or kept in, to direct matrix assisted laser desorption time of flight mass spectrometry and detection of spectral masses between 2,000 and 100,000 m/z. The changes in spectral masses, leading to changes in the profile of the spectra, are characteristic of disorders of fetal development and pregnancy.

### BACKGROUND

Maternal fetal wellbeing (obstetric care) is central to many world National healthcare services. The increased medicalization of pregnancy from assisted reproduction to emergency delivery due to complications of the pregnancy, such as eclampsia or fetal abnormality/distress, means that a significant proportion of any such budget is spent on Maternity and Reproductive Health. For example in the UK 3.2% of the National Health Service Budget (£3.44 billion in 2010/11) is currently spent on assisted reproduction and obstetric care.

From an emotional and financial perspective (a complicated pregnancy and delivery costs an individual household 10 to 100 times more than that of an uneventful pregnancy), testing to see if the baby is “normal”, progressing and “unharmed” is now an expectation of a healthcare service. The rise in GDP expenditure in healthcare over the last 50 years has been due in part to increased testing including that now associate with maternity. Alongside increased accuracy of such tests, there has been exponential rise in costs which have largely been ignored. There is now a need to develop pregnancy screening tests that have high efficacy but affordability to end users be they national health services, health insurance companies or individuals.

### Gestational Trophoblastic Diseases

Gestational Trophoblastic Diseases (GTD) covers a wide range of pregnancy-related disorders that are in many ways cancerous in nature but can also be considered forms of pregnancy with a spectrum of additional clinical features, morphologic characteristics and sometimes pathologies. GTDs were first described around 400 BC by Hippocrates but finally linked to pregnancy in 1895. GTDs present themselves as premalignant disorders of complete and partial hydatidiform mole or malignant cases such as invasive mole, choriocarcinoma, and the rare placental-site trophoblastic tumour (PSTT). Malignant GTD can be referred to as gestational trophoblastic tumours or neoplasia (GTT or GTN). The initial manifestations of GTD include, vaginal bleeding and vomiting. However, women with gestational trophoblastic tumours present with heavier abnormal vaginal bleeding, and amenorrhea, specifically in placental-site trophoblastic tumour.

As in normal pregnancy hCG tests form a major part of the diagnosis and monitoring of GTD. For many obstetricians a positive pregnancy test in the absence of a viable pregnancy (as confirmed by ultrasound) is immediately indicative of a potential GTD. In some cases, in particular choriocarcinoma, the tumour can be highly invasive and a rapid intervention by an oncologist is essential to preserve the chances of survival for the patient.

However, the management of GTD with hCG tests and ultrasound alone is insufficient to provide the full picture of varied and complicated disorders within the spectrum of GTD. Problems have already been identified in hCG assays which do not discriminate (or discriminate to much) the forms of hCG which can be detected and that in some cases false positives and false negatives have led to misdiagnoses and needless therapies. These issues have led to a better defining of hCG assays in terms of the epitopes recognized in each assay, but it has not circumvented the root problem underlying the metabolic differences resulting in the production of hCG variants. It has been suggested that the hCG form is of more significance than the absolute amount of total hCG. This qualitative problem needs a qualitative solution.

### Hyperemesis Gravidarum

Hyperemesis gravidarum is defined as persistent vomiting in pregnancy resulting in weight loss, (>5% body mass in the USA, as defined by the ACOG, or >15% body mass in the UK, defined by the RCOG), combined with ketosis, hypokalemia and dehydration. Therefore, it is not simply an exaggerated form of the classic ‘morning sickness’ which accompanies early pregnancy. Hypovolemic and metabolic derangement, especially that of thyroid function, are potentially life threatening to both the mother and developing fetus. In contrast, the experience of mild and self-limiting nausea and vomiting (morning sickness) are common occurrences in up to 80% of all women during pregnancy.

Early diagnosis of Hyperemesis Gravidarum is clinically important due to the potential harm, as if left unmanaged these symptoms may worsen significantly, leading to loss of the fetus and in some cases result in maternal fatality. Mortality rates from hyperemesis were considerable and as high as 159 deaths per million births, before the introduction of intravenous rehydration therapies. In the United States, the incidence of hyperemesis is much higher than in the UK, and represents an annual hospital admission of 38,000 women.

There is no universally accepted explanation or cure for hyperemesis, with management based on symptomatic approaches, including therapies such as intravenous fluid and electrolyte replacement, thiamine supplementation and psychological support. Nevertheless, early detection and intervention of the disorder is believed to improve management and a biochemical marker in this regard would be useful.

Although no direct link has yet been established, it has often been suggested that hCG could be the most likely cause of hyperemesis gravidarum during pregnancy. This is especially true in cases of women at risk of gestational trophoblastic diseases where hCG levels tend to be unexpectedly high. Older studies revealed that in 36% of the hyperemesis cases examined hCG levels were above the 97.5 percentile of the normal value; however, simply having high hCG is not always consistent with hyperemesis.

Biochemical hyperthyroidism, associated with hyperemesis gravidarum, has been attributed to a mechanism involving hCG cross-reacting agonistically with the thyroid stimulating hormone (TSH) receptors and is identified as a risk factor of hyperthyroidism. Since, the alpha subunits of both TSH and hCG are identical they possess some agonistic effect especially at high circulating concentrations. These thyrotrophic effects of hCG are known to manifest as severe vomiting and nausea, which is the chief symptom of hyperemesis gravidarum; so much so that historically thyroid function tests were a determinant factor in pregnancy diagnosis and patient management. Furthermore, acidic isoforms of hCG have been speculated to be a causative factor in protracted cases of severe vomiting and nausea. The underlying explanation is that that acidic forms of hCG, due to the presence of additional sialic acid rich glycosylations, promotes a longer circulating half-life of the hormone, and thus an exacerbation of the thyrotrophic effects described. Significantly recent studies indicated that the involvement of hCG in hyperemesis might be structural rather than simply quantitative and a thyrotropic form of hCG has been suggested.

### Pre-Eclampsia

Pre-eclampsia is a disorder of pregnancy characterized by high blood pressure and large amounts of protein in the urine. This can lead to impaired liver function, kidney dysfunction, pulmonary oedema, and neurological and visual disturbances. If left untreated, preeclampsia can develop life-threatening occurrence of seizures during pregnancy, termed eclampsia. Even if detected early pre-eclampsia is associated with multiple maternal and fetal adverse effects. Affecting between 2-8% of pregnancies worldwide, it may develop from as early as 20 weeks of gestation, though most commonly after 32 weeks. Pre-eclampsia occurring before 32 weeks is considered early-onset and is associated with increased morbidity. Most cases are diagnosed before term and delivery of the fetus and placenta is the only known treatment for pre-eclampsia.

The pathogenesis of preeclampsia has not been determined although abnormal placentation is a strong predisposing factor. There are a host of contributing and related factors that include maternal immunological responses. Central to the effects of preeclampsia are the resulting presence of utero-placental hypoxia, an imbalance in angiogenic and anti-angiogenic proteins, oxidative stress, maternal vascular endothelial dysfunction, and elevated systemic inflammation. Given the multi-factorial nature of the disease, it is not yet possible to routinely predict preeclampsia.

### Multiple Pregnancy.

A multiple pregnancy occurs when the women is carrying more than one embryo or foetus, such a twins, triplets, quadruplets etc. Detecting a multiple pregnancy is usually established in the weeks 10-13 of pregnancy following demonstration of multiple embryonic sacs by ultrasound. The analysis of protein markers to indicate pregnancy disorders have been used for decades but proteomic analysis of the serum and urine are relatively new approaches especially to identify twins.

### Spontaneous Abortion or Miscarriage

Spontaneous abortion (also known as miscarriage) is a common complication of early pregnancy and occurs when the embryo or foetus dies naturally within the first 20 weeks of pregnancy. Among women who know they are pregnant, the miscarriage rate is approximately 10% to 20% while rates among all conceptions is around 30% to 50%. About 5% of women have two miscarriages in a row, but women can have multiple miscarriages. Knowing about this early could offer obstetricians an opportunity to intervene and potentially reduce the risk of losing the pregnancy.

### Embryo/Early Pregnancy

Human chorionic gonadotrophin (hCG) is one of the most widely studied markers in embryonic development. Despite its worldwide usage as an obstetric marker, hCG is often regarded as little more than a signal for maternal recognition of pregnancy through the subsequent maintenance of progesterone production. The hCG molecule has several isoforms attributed to varying degrees of glycosylation and stages of metabolic degradation, some of which may now have more specific functions different to that of “normal” hCG. The normal or regular or intact hCG (as it is often referred, and subsequently indicated as hCG in this example) is the predominant form of hCG produced by the trophoblast during pregnancy, and is a heterodimer consisting of a hCGα, and hCGβ subunit; it is also often erroneously referred to as beta hCG and for the purpose of this example needs clarification, the beta subunit of hCG is distinct.

Hyperglycosylated hCG (hCGh or invasive trophoblastic antigen, ITA) is a form of hCG produced by during pregnancy especially during the first trimester. hCGβ is the free form of the hCGβ subunit arising either through dissociation of hCG or through ectopic expression in the absence of hCGα. There have been numerous studies on the expression and production of hCG by developing embryos in culture, however few have linked the production of specific hCG isomers to particular function or dysfunction, and only more recently suggested that variability of hCG concentrations in vitro may act as a marker for implantation potential. Several studies have suggested that measuring hCGh on its own can provide an accurate prediction of pregnancy failures. Others reported that hCGh can be used to identify a term pregnancy against failing pregnancy on the day of implantation with high accuracy. Thus, it can be useful to identify this hCG isoform in order to differentiate a successful pregnancy from that of a potential failure. It has also been noted that a correlation exists between early embryo proteomic, secretomic, and metabolic profiling, and successful implantation rates. Therefore, hCG isoform profiling in the secretome may be the key for identification of viable embryos, particularly in a non-invasive manner. This is important, considering that despite recent studies suggesting several metabolic and biochemical markers for predicting implantation, none have yet been found of any clinical value.

US2009/0075293 describes a method for predicting embryo viability by identifying the presence or absence of one or more pregnancy associated markers such as molecular isoforms of hCG in a sample. High quality embryos were distinguished from low quality embryos based on the embryo's ability to utilize/metabolise proteins in the molecular weight range 9149-9157, thought to be an isoform of the CRAb protein. The samples were processed prior to analysis, by treatment with DTT and the removal of albumin. The method is based on identifying the presence or absence of certain proteins. The method of the present invention does not require the identification of specific proteins. The present invention looks for changes in the overall profile of the spectra, or in certain regions as compared to the spectra obtained from an equivalent sample from healthy ongoing pregnancies

The method herein describes rapid screening of samples obtained from a pregnant woman, subjected to direct mass spectral analysis, such as MALDI-ToF Mass spectrometry. In addition analysis of fluid (buffered saline, blastocyst culture media) that surround an embryo in culture during assisted reproductive technology procedures are similarly analyzed by direct mass spectral analysis, such as MALDI-ToF mass spectrometry. Analysis may be carried out directly on a sample or following dilution, for example in distilled deionised water, or other suitable diluent. Optionally dilution at the range of 1/2 to 1/1000 in for example distilled deionised H₂O or 0.1% trifluoroacetic acid (TFA) in distilled deionised H₂O may be necessary for concentrated samples. The resulting spectra are examined as charged ions at the Mass/charge range of at least 2000 m/z to 100,000 m/z or higher. Changes in the profile of the spectra as a whole, or certain regions or combinations thereof, as opposed of the presence/absence of certain peaks, are compared to the spectra obtained from equivalent sample from healthy ongoing pregnancies. This provides a prognosis for pregnancy. It is not necessary to look for any certain known proteins, or to identify the proteins which cause the change in profile.

The present invention provides a method of providing a prognosis for a pregnancy, comprising subjecting a sample to direct mass spectral analysis. The spectra resulting from said analysis can be compared to the spectra obtained from normal pregnancies.

“Direct mass spectral analysis” means that the data generated from the mass spectral analysis is used in the method, and not the inferred mass of the components present in the sample.

The sample can be a sample obtained from a pregnant woman, or a sample of the culture and/or storage media surrounding an embryo.

“Embryo” as used here in refers to any cell or group of cells post fertilization, but prior to intrauterine transfer. It includes zygotes, blastomeres, morulae, and blastocysts.

“Prognosis for a pregnancy” as used herein refers to predicting the likelihood of an embryo implanting successfully, as well as, diagnosing or predicting the likelihood, i.e. providing a prognosis of a pregnant woman suffering from a disorder of pregnancy, or having a multiple pregnancy. The method can also be used to detect embryos with chromosomal abnormalities. A prognosis for pregnancy can be a successful ongoing pregnancy or a failing pregnancy. As used herein an “ongoing” pregnancy is a normal pregnancy that is expected to proceed to term and result in a live birth. A “failing pregnancy” as used herein refers to a biochemical pregnancy, or a pregnancy resulting in a spontaneous abortion/miscarriage, recurrent miscarriage,

The method can be used to provide a prognosis and/or diagnosis of a disorder of pregnancy, as well as a prognosis for a multiple pregnancy. For these methods the sample is obtained from a pregnant woman. As used herein a “disorder of pregnancy” includes Ectopic pregnancy, biochemical pregnancy, early pregnancy loss (EPL), Threatened Miscarriage, Spontaneous abortion or miscarriage, Hyperemesis Gravidarum and Gestational Trophoblastic Diseases, Placental Insufficiency, Pre-eclampsia, Gestational Diabetes, Placenta previa, placenta accreta, placenta percreta, Obstetric Cholestasis, and Recurrent Miscarriage in both normal and assisted reproduction. Gestational Trophoblastic Diseases include molar pregnancies, choriocarcinoma, and placental trophoblastic tumour (PSTT). The term “disorder of pregnancy” as used herein, when referring to a sample obtained from a woman (i.e. not an embryo culture fluid sample) excludes conditions within the fetus such as fetal aneuploidy.

The method of the invention provides a method of screening for pregnancy disorders which are already present when the sample is obtained such as Ectopic pregnancy, Threatened Miscarriage, Hyperemesis Gravidarum and Gestational Trophoblastic Diseases, as well as multiple pregnancy. The method of the invention can also provide an indication of the risk of developing other disorders of pregnancy which generally occur later in the pregnancy (i.e. after the sample has been taken) such as Placental Insufficiency, Pre-eclampsia, Gestational Diabetes, Obstetric Cholestasis, Recurrent Miscarriage, and spontaneous abortion in both normal and assisted reproduction. Thus the method has both diagnostic and prognostic value. Preferably the disorder of pregnancy is selected from Pre-eclampsia, Hyperemesis Gravidarum, or Gestational Trophoblastic Diseases. In one embodiment the disorder of pregnancy is not a Gestational Trophoblastic Disease. Preferably the method is used to provide a prognosis of a multiple pregnancy or a spontaneous abortion.

A “multiple pregnancy” as used herein refers to a pregnancy where the woman is carrying more than one embryo or foetus, such as twins, triples, quadruplets, quintuplets, etc.

A “biochemical” pregnancy as used herein refers to the situations where a pregnancy test provides a positive result, but the embryo fails to implant properly, so does not result in an ongoing pregnancy.

The sample obtained from a pregnant woman is a bodily fluid sample. Bodily fluids include cerebrospinal fluid, seminal fluids, vaginal fluids, interstitial fluids, tissue aspirates, saliva, and urine such as early morning and midstream urine samples, blood and serum. The sample may be a spot card sample, for example a urine or blood spot card sample, wherein the sample is applied to filter paper or other capture material, allowed to dry and stored for future analysis. The blood sample can be a whole blood sample collected using conventional phlebotomy methods. For example, the sample can be obtained through venupuncture or as a pin prick sample, such as a finger-stick or heel prick. The blood sample may be a dried blood spot captured on filter paper or other suitable blood spot capture material. Preferably the sample is a urine sample.

The sample from a woman may be obtained at any point during the pregnancy. In one preferred embodiment the sample may be obtained from a subject in the first trimester of the pregnancy, for example 4-13 weeks gestation, preferably 6-11 weeks gestation, 5-7 weeks gestation and/or 8-11 weeks gestation. The sample may also be obtained during the second trimester or later, i.e. 14 or more weeks gestation, preferably 16-35 weeks, more preferably 18-30 weeks gestation.

Alternatively the method can be used to give a prognosis of pregnancy, such as successful implantation or outcome, for a cultured embryo, provided by assisted reproduction. The method can give a prognosis for a successful implantation followed by an ongoing pregnancy or a failing pregnancy such as a spontaneous abortion (miscarriage), a biochemically positive pregnancy, as well as detect chromosomal abnormalities in the embryo. The method can be carried out on a sample of the fluid (such as buffered saline, embryo or blastocyst culture media) that surrounds an embryo in culture during assisted reproductive technology procedures. This includes the fluid in which the embryo is stored or frozen. It also includes any fluid arising in the culture media from the embryo itself, for example after blastocyst hatching. The sample can be taken at any time prior to transfer. Preferably the sample is taken 3-5 days post-fertilization. The sample can be taken either prior to, or after blastocyst formation. The sample can be taken shortly i.e. 0.5-12 hours, before transfer to the recipient.

“Chromosomal abnormalities” as used herein refer to any condition where the cell has a variation from the normal chromosome number e.g. 46 in humans. These include fetal aneuploidies, such as Down's Syndrome, Patau syndrome Turner Syndrome, Klinefelter syndrome, Edwards syndrome and triple-X as well as fatal trisomies of any other of the 23 chromosomes.

The sample (either maternal or embryo culture fluid) may be a neat sample. Alternatively, the sample may be diluted or processed (concentrated, filtered, etc.).

Preferably the sample (either maternal or embryo culture fluid) is diluted. The sample may be diluted 1/2 (i.e. one part sample in 1 part diluent), 1/5, 1/10, 1/100, 1/200, 1/500, 1/1000, 1/2500 or more. Most preferably the sample is diluted 1/1000 i.e. one part sample in 1000 parts diluent. Preferably the diluent is water or 0.1% trifluoroacetic acid (TFA) in distilled deionised H₂O, more preferably distilled deionized water.

If the sample is stored on a spot card or blood spot capture material, it can be reconstituted using a suitable buffer, or a diluent. Preferably the diluent is water or 0.1% trifluoroacetic acid (TFA) in distilled deionised H₂O, more preferably distilled deionized water.

Preferably the sample (either maternal or embryo culture fluid) is not processed prior to dilution. Such processing includes concentrating the proteins of interest e.g. hCG; isolating hCG by for example HPLC, removal of contaminating proteins e.g. albumin, protease e.g. trypsin digestion or treatment with a chemical agent to disrupt or break intramolecular bonds. In particular, the sample is preferably not treated with a reducing agent. More preferably the sample is not treated with dithiothrietol (DTT). The sample is preferably not treated with DTT if it is a urine sample.

During direct mass spectral analysis, a spectra is generated using a matrix. Suitable matrix compounds include sinapinic acid, ferulic acid (FA) and alpha 4-cyano hydroxycinnamic acid (CHCA). The intensity of the characteristic resolved mass peaks are measured as specific m/z values ranges or a ratio determines the relative abundance of specific m/z peaks. The spectra is analyzed over the range of 1000 m/z to 100,000 m/z. Preferred ranges include 2000 m/z to 100,000 m/z, 4,000 m/z-35,000 m/z, 4,000 m/z-80,000 m/z, 10,000 m/z-75,000 m/z, 70,000 m/z-80,000 m/z 2,000 m/z-14,000 m/z; 6,000 m/z-14,000 m/z, 6,000 m/z-8,000 m/z, 12,000 m/z-50,000 m/z 20,000 m/z-50,000 m/z; 22,000 m/z-26,000 m/z; 30,000 m/z-40,000 m/z; and 35,000 m/z-40,000 m/z, or combinations thereof.

The spectra as a whole can be analysed, or one or more subranges, or regions can be analysed e.g. 2,000 m/z to 16,000 m/z, and 70,000 m/z-80,000 m/z Thompson Units (Th) identify a region of mass:charge (m/z) which is itself proportional to mass in kDa.

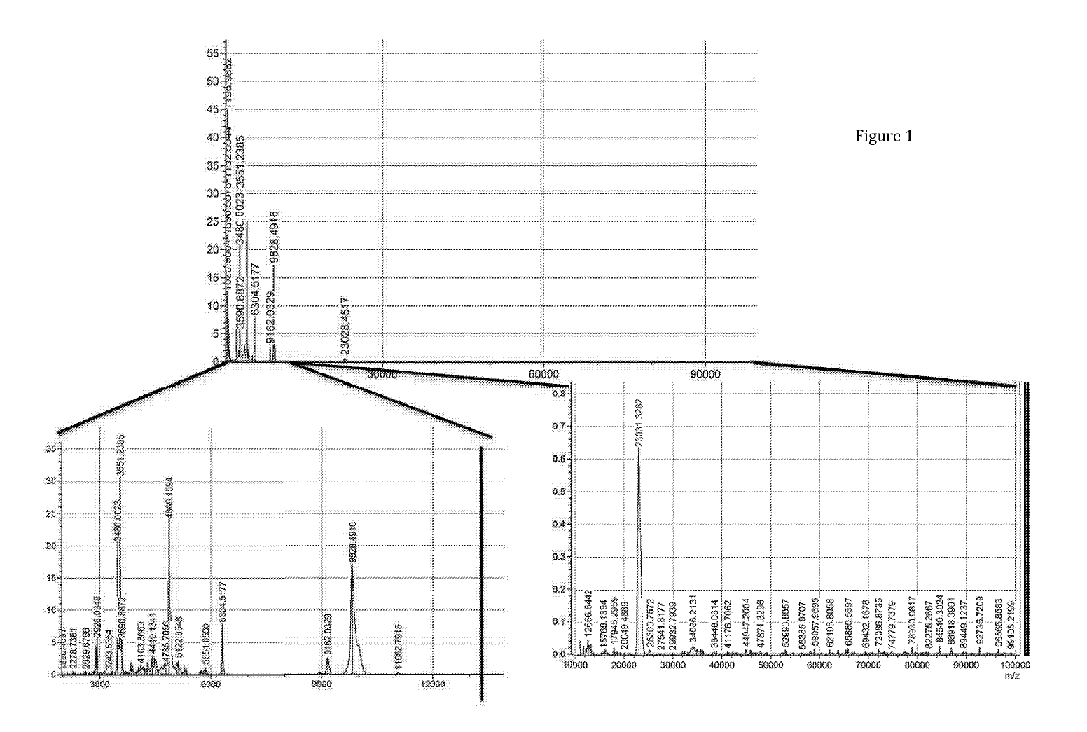

Clusters of peaks are usually seen in urine, as shown in FIG. 1, at:

2,000 m/z to 6,000 m/z—predominately, but not exclusively, arising from metabolites of trypsin inhibitors.

6,000 m/z to 14,000 m/z—predominately, but not exclusively, arising from the beta core metabolite of human chorionic gonadotrophin.

14,000 m/z to 100,000 m/z predominately, but not exclusively, molecular variants of hCG and its alpha and beta subunits.

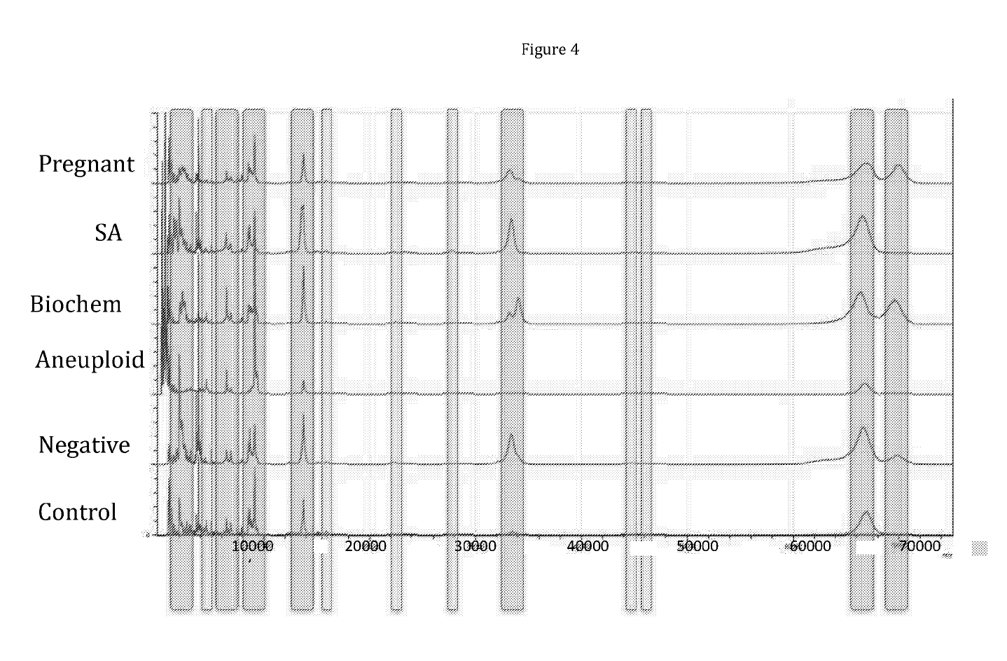

Peaks usually seen in embryo culture fluid vary between different pregnancy outcomes as shown in FIGS. 4 (and 9).

Methods of generating mass spectra, such as mass spectrometry including MALDI-Tof MS, are commonly not quantitative techniques. For example the Y axis in these spectra is an indicator of “relative strength” of mass peak within the spectra, but not between mass peaks in one sample versus another sample. In order to overcome this, normalization needs to render Y axis value comparable between sample spectra. Thus the spectra obtained from the direct mass spectral analysis are preferably normalized. The spectra are subjected to data processing which results in a normalized statistically determined index of relative proportion of mass spectra. This converts the qualitative mass spectra into a quantitative value. Normalization is the process of producing a data structure to reduce repetition and inconsistencies of data. Several normalization techniques are possible. Typical normalization methods include percentage of total area at a given point, Square difference and ratio of differences. The percentage difference is calculated as

Percentage difference=(Y1−Yref/Y ref×100%)

Wherein Y ref is the minimum measured Y value of the spectra, and Y1 is Y value for each point.

The square difference is calculated as

Square Difference=(Y1−Y ref)²

The ratio difference is calculated as

Ratio Difference=(Ratio 1−Ratio 2).

Thus the data from the mass spectra is manipulated (normalized) in order to provide a quantitative measure of the qualitative change shown on the spectra.

Preferably, the spectral model is created by a method of data processing which results in a normalized statistically determined index of relative proportion of mass spectra within a set range. This renders all spectra comparable such that the median and centile variability at any given mass value can be modeled.

A normalized statistically determined index of relative proportion of mass spectra within a given range can be calculated from using the total area under the curve of the mass spectra in a defined area of interest or combinations thereof e.g. 2000-14000 m/z; 6000-14,000 m/z; 12,000 m/z-50,000 m/z, 30,000 m/z-40,000 m/z and 14,000 to 100,000/m/z. This can then be used to calculate the relative intensity of mass regions that alter in samples from patients with particular disorders.

The area under the curve of mass spectra is calculated by dividing the mass spectra into a plurality of bins of a given number of m/z. As used herein “Bin” has its usual statistical meaning, for example, of being one of a series of ranges of numerical value into which data are sorted in statistical analysis. For example the bins can be 100 m/z, 50 m/z, 25 m/z, 10 m/z, 5 m/z or 3 m/z in size. The smaller the size of the bin used, the more refined the method. If two or more regions of interest within the spectra are analysed, then different sized bins can be used for reach region. For example the range 1,000-10,000 m/z can be analysed using 5 m/z bins together with the range 14,000-100,000 m/z analysed using 10 m/z bins.

The relative intensity (Y Axis value) can be calculated by the “square of difference” method and therefore a comparable Y value given for every bin. In this method, the minimum Y value of the spectra (Y ref) was subtracted from the Y value at every bin and the difference was squared. The formula used to calculate square of difference=(y1−yref)² and the calculated square of difference was then named as “relative intensity”.

The relative intensity at each mass bin in a sample can be captured using commercially available statistical tests such as MATLAB®, Stats Direct™ and Origin 8™.

Preferably, each sample is compared against a reference spectral model. The “reference spectral model” is the expected mass within a set range, determined from statistical analysis of a collection of samples obtained for normal healthy pregnancies. As used herein a “normal healthy” pregnant woman is one who does not have a pregnancy disorder, or woman who has an ongoing pregnancy following successful implantation of a cultured embryo. Preferably the reference spectral model of expected mass is determined from statistical analysis of a collection of samples at matched gestational age. The sample is compared to a reference spectra generated from the same sample type e.g. a culture fluid sample is compared to a reference spectra generated from embryo culture fluid, and not a maternal sample, and vice versa. Any changes, such as a change in mass, an increase or decrease in the relative intensity, or a change in the ratio of relative intensity between two or more peaks in the sample spectra as compared to the reference spectra may be indicative of a change in the prognosis for pregnancy. For example, an increase in the relative intensity of a peak associated with a disorder of pregnancy may be indicative of the disorder of pregnancy being present. Preferably the range is between about 500 m/z-100,000 m/z, for example 1,000 m/z-75,000 m/z, 2,500 m/z-50,000 m/z, 5,000 m/z-25,000 m/z, 6,000 m/z-14,000 m/z or 12,000 m/z-50,000 m/z or combinations thereof. Most preferably the range is 6,000 m/z-14,000 m/z. Preferably the spectral model of expected mass is determined from statistical analysis of a collection of samples of the same type e.g. maternal or embryo culture fluid at matched gestational or embryo age.

Preferably, the spectral model is created by a method of data processing which results in a normalized statistically determined index of relative proportion of mass spectra within a set range. This renders all spectra comparable such that the median and centile variability at any given mass value can be modeled.

Preferably, a parallel “disease” model, as generated above from normalized statistically determined index of relative proportion of mass spectra within a set range is created from samples obtained from a pregnant woman with a disorder of pregnancy, or culture fluid samples from embryos which failed to successfully implant. The spectra from a sample can then be compared to the disease model generated from samples of the same type. The presence of a change in mass or peak associated with a disorder of pregnancy or an embryo less likely to implant may be indicative of a disorder of pregnancy or poor prognosis for implantation and/or pregnancy.

Once the spectra has undergone a method of data processing which results in a normalised statistically determined index of relative proportion of mass spectra any significant changes in mass can be attributed to a given disorder or diagnostic utility.

The reference spectral model and the disease model, are then compared by plotting in order to identify ‘hot spots’ i.e. points or regions of difference between the two models(for example as shown in FIG. 4). This may be a decrease or increase in the size of a peak, or the appearance of a peak. The points of difference can then be used to determine the prognosis of pregnancy. This may be done by using a suitable algorithm.

Preferred ranges and combinations thereof for analysis in various conditions are provided below:

Gestational trophoblastic disease (GTD): 35,000-45,000 m/z, preferably 37,600-39,600 m/z

Hyperemesis gravidarum: 22,000-40,000 m/z; preferably 11,000-26,000 m/z, and/or and/or 32,500-34,500 m/z, and/or 35,000-37,000 m/z and/or 37,500-40,000 m/z.

Pre-eclampsia: 2,000-100,000 m/z; preferably 14,000-100,000 m/z and/or 2,000-14,000 m/z; especially 6,000-8,000 m/z.

Successful of embryo implantation: 22,000-40,000 m/z; preferably 22,000-26,000 m/z and/or 35,000-40,000 m/z.

Failing implantation: 9700-10,000 m/z and/or 7,500-8,000 m/z and/or 10,500-12,000 m/z.

Outcome of pregnancy from embryo culture fluid: 12,000-50,000 m/z. Preferably 4600-4700 m/z and/or 6400-6500 m/z and/or 9100-9200 m/z. Alternatively preferred range include 15,800-16,100 m/z and/or about 36,400 m/z and/or 9,000-9500 m/z, and/or 8,400-8,700 m/z and/or about 28,000 m/z.

Spontaneous abortion: 6000-12000 m/z. Preferred ranges include 6000-6050 m/z and/or 6450-6500 m/z and/or 9350-9400 m/z and/or 11150-11200 m/z and/or 11800-11850 m/z.

Multiple gestations: 6000-14000, preferably 9000-13000. Other preferred ranges include 12500-12550 m/z and/or 10300-10350 m/z and/or 9600-9650 m/z.

For all of the conditions listed above analysis can be carried out within a single range or a combination of any of the ranges listed.

The method can be used to detect chromosomal abnormalities such as aneuploidy in an embryo by analyzing the embryo culture fluid. Current methods for detecting chromosomal abnormalities in embryos require removal of a cell from the developing embryo which is then used for genetic analysis. This can be PGS (put in the full name) where simple karyotyping is carried out to identify the number of chromosomes present and thus aneuploidy. Alternatively genetic conditions can be identified by looking for sequence abnormalities. The method of the invention allows chromosomal abnormalities to be identified from the culture fluid, and thus eliminates any risk of harm to the embryo, as it is not necessary to remove a cell.

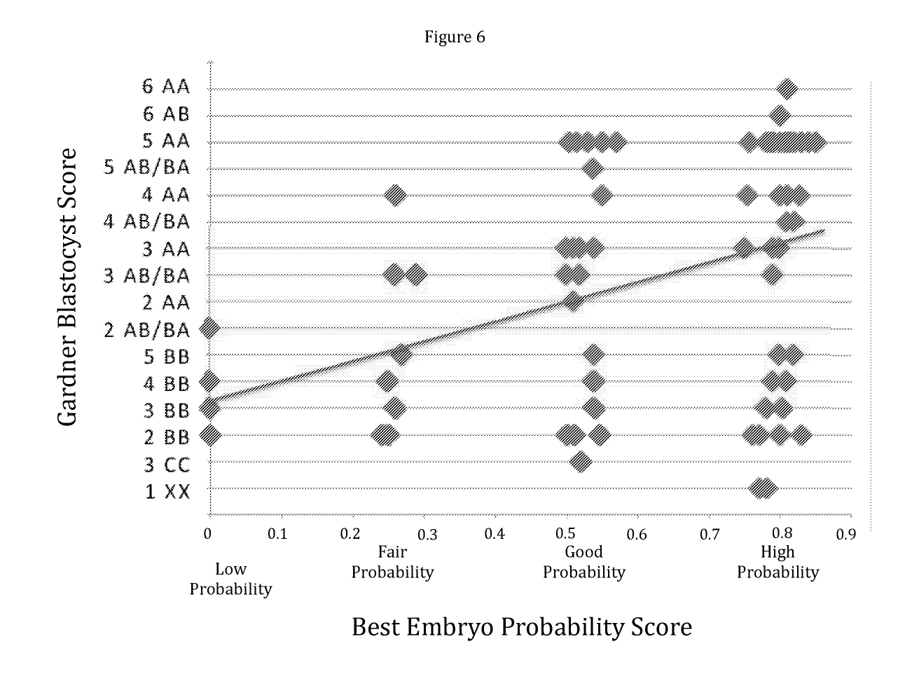

Preferably, the method is used to provide a prognosis for successful embryo implantation, prior to embryo transfer. The likelihood of an embryo successfully implanting after transfer can be assessed by analysing the spectra in the range 22,000 m/z-40,000 m/z from a sample of culture fluid surrounding the embryo. The presence of abnormal peaks in the ranges about 22,000 m/z to 26,000 m/z and about 35,000 m/z-40,000 m/z in a culture fluid sample may be indicative of an embryo which is less likely to implant successfully. The sample is preferably obtained at day 3-5 post fertilisation. The results obtained can be used to score the embryo based on the likelihood of successful implantation, resulting in an ongoing pregnancy. The results obtained using the method of the invention are improved compared to the conventional (Gardner scoring) methods (see FIG. 6). In addition reliable results can be obtained by the method of the invention at an earlier stage e.g. day 3 post fertilisation as opposed to day 5, immediately prior to transfer. This provides more time for decision making and embryo selection.

Preferably, the method is used to provide a prognosis for successful embryo implantation, after embryo transfer. Changes in mass within a urine sample from a woman post embryo transfer can also indicate whether an embryo has successfully implanted. Urine samples from women post embryo transfer which have a reduction or elimination of peaks in the range about 9,700 m/z to 10,000 m/z, and/or abnormally elevated peaks around about 7,500 m/z to 8,000 m/z and about 10,500 m/z to 12,000 m/z, are indicative of failing implantation.

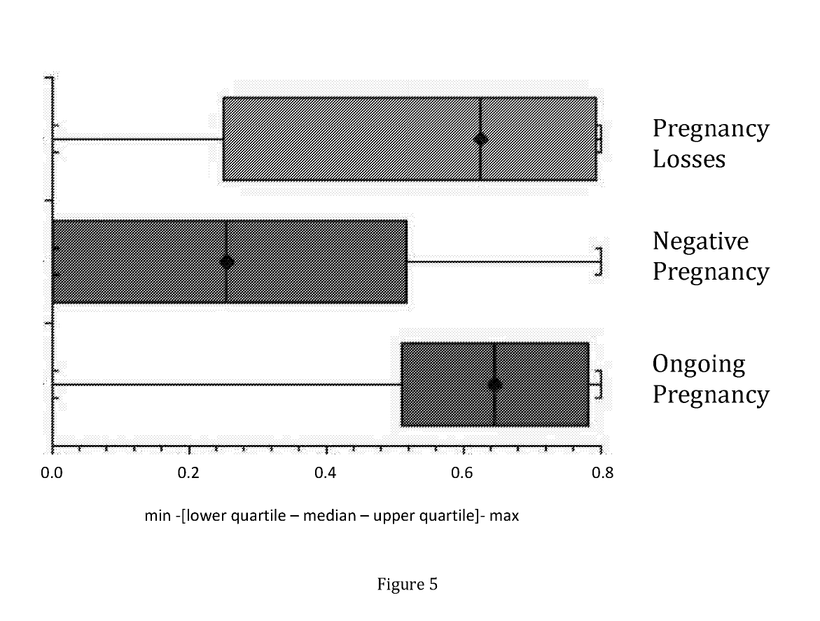

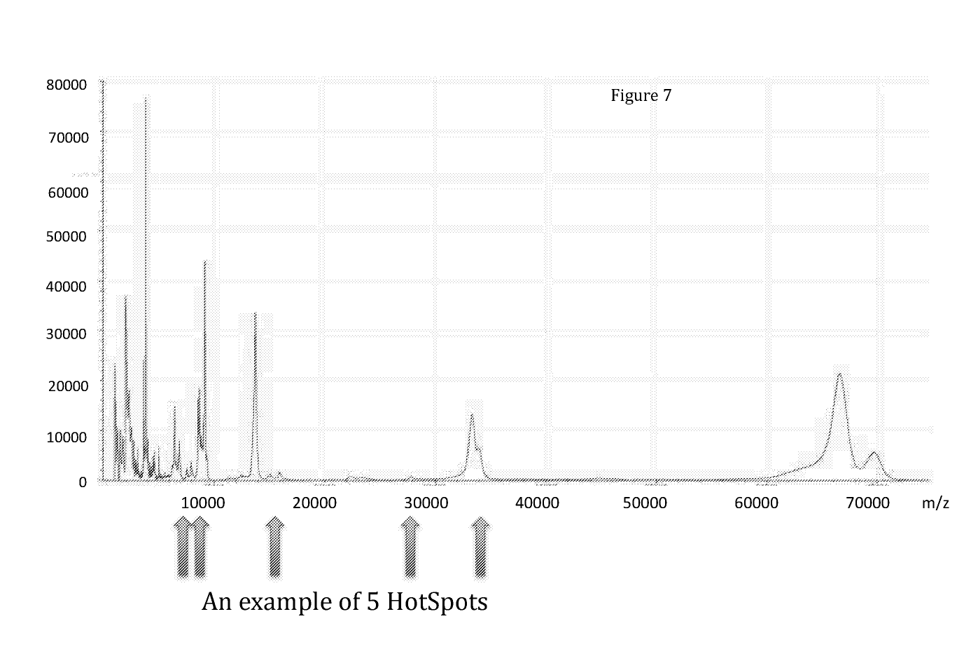

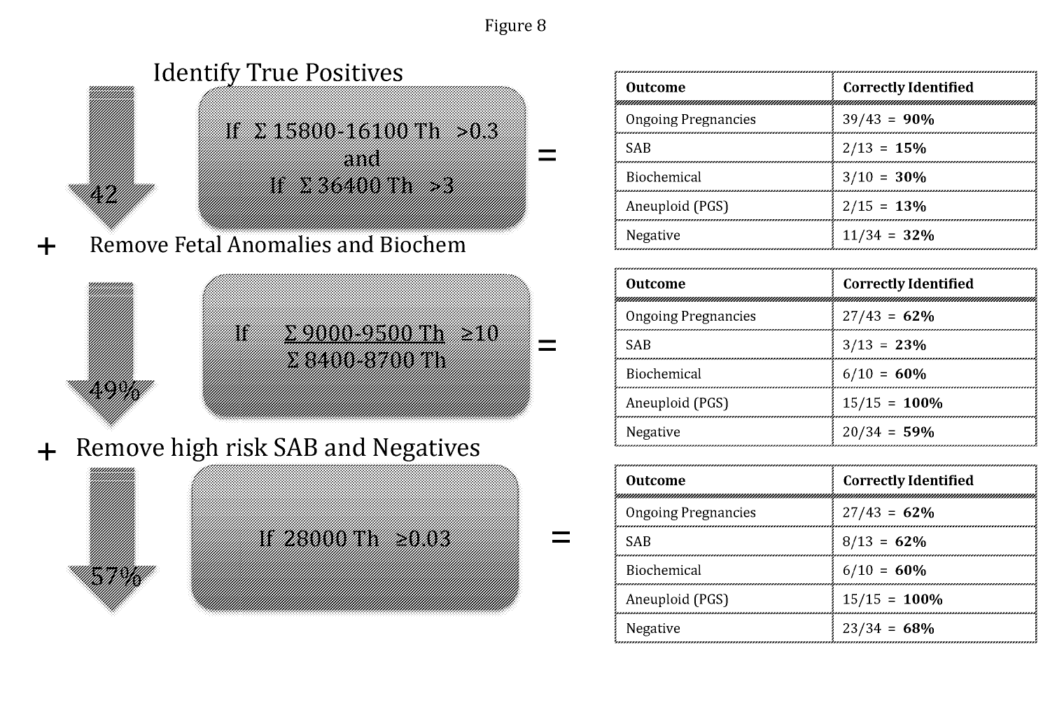

The specific regions from 4600 to 4700 m/z, from 6400 to 6500 m/z and from 9100 to 9200 m/z were identified as areas of mass spectral differences between samples, or “hot spots” which can be used to provide a prognosis of different outcomes of pregnancy in the culture fluid surrounding embryos. (See FIG. 5). In addition areas of difference can be used to create a Predictive Algorithm. As shown in FIGS. 7 and 8 hot spots can be identified in the spectra, and then these areas of difference can be used to build an algorithm which can be used to provide a prognosis of pregnancy. Peaks in the ranges about 15,800 m/z to 16,100 m/z, about 36,400 m/z, about 9,000-9,500 m/z, about 8400 m/z-8700 m/z and about 28,000 m/z and combinations thereof in a culture fluid sample can be used in to develop an algorithm. Methods of generating a suitable algorithm are known to the skilled person.

Preferably, the method is used to detect multiple gestations by analysis of a maternal sample. Detecting a twin or triplet pregnancy is usually established early in pregnancy following demonstration of multiple embryonic sacs by ultrasound. The analysis of protein markers to indicate pregnancy disorders have been used for decades but proteomic analysis of the serum and urine are relatively new approaches especially to identify twins. The method of the invention can be used to correlate the spectral analysis of urine by MALDI ToF MS to identify multiple from singleton pregnancies.

The preferred range for analysis for the detection of multiple gestations is 6,000 m/z-14,000 m/z. Peaks in the ranges about 10,300 m/z to 10,350 m/z, about about 9,600-9,650 m/z, and about 12500 m/z-12550 m/z and combinations thereof in a maternal sample can be used in to develop an algorithm or decision tree. Changes in the protein profile as compared to a normal pregnancy occur in both ranges.

Preferably, the method is used to provide a prognosis for and/or detect pre-eclampsia by analysis of a maternal sample. The preferred range for analysis for the diagnosis and/or prognosis of pre-eclampsia is 14,000 m/z-100,000 m/z and also in the low mass range of 2,000 m/z-14,000 m/z, preferably 6,000-8,000 m/z. Changes in the protein profile as compared to a normal pregnancy occur in both ranges.

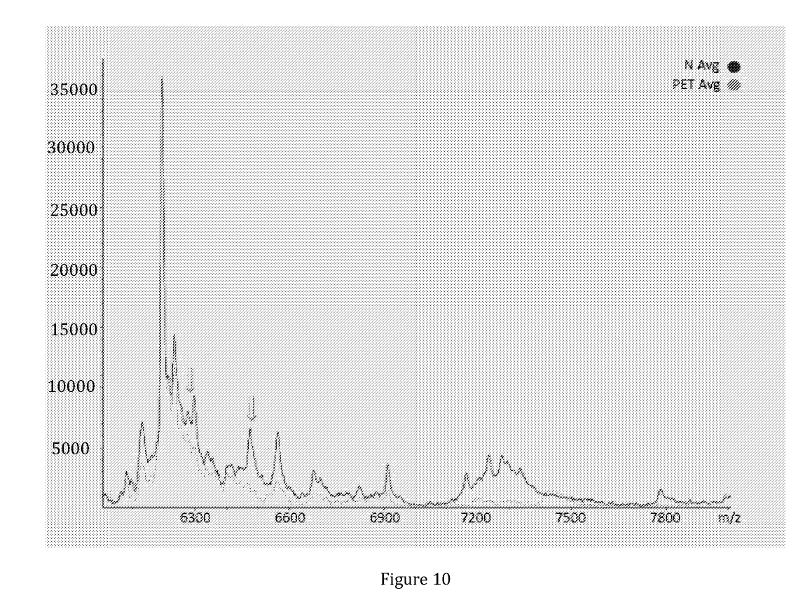

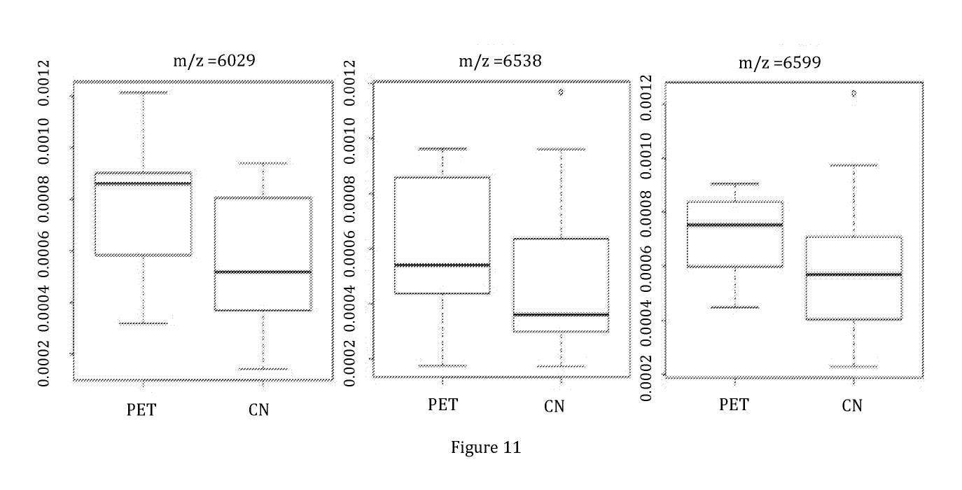

Preferably a mass shift in the 2,000 m/z-14,000 m/z range in a sample taken in the first trimester is indicative that pre-eclampsia is likely to develop at a later stage. Changes in the peaks at 6029 m/z, 6538 m/z and/or 6599 m/z±5 m/z are indicative of pre-eclampsia as shown in FIGS. 10 and 11. Therefore a positive result is indicative that the patient should be given prophylactic treatment, e.g. aspirin and low molecular weight heparin.

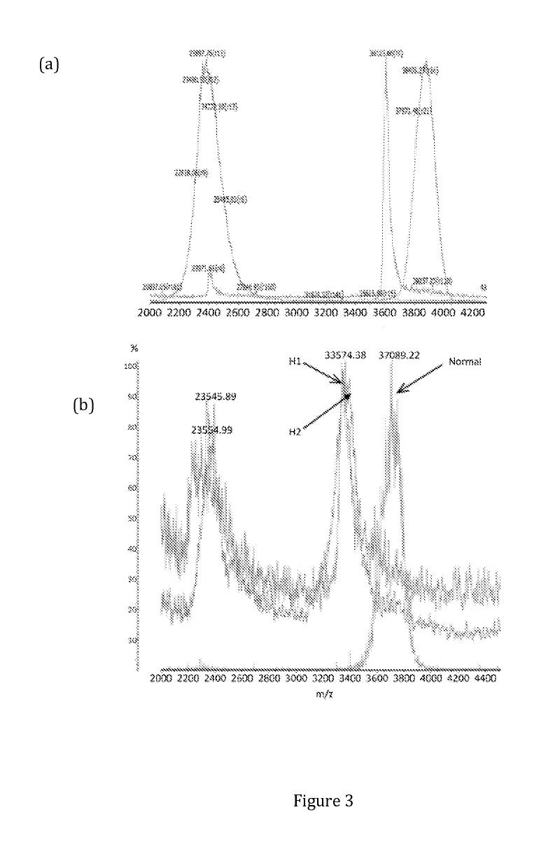

Preferably, the method is used to detect the presence of Hyperemesis gravidarum from analysis of a maternal sample. For Hyperemesis gravidarum the preferred range for analysis is 22,000 m/z to 40,000 m/z. In samples from women with a normal pregnancy peaks are seen within the range 22,000 m/z to 26,000 m/z, 35,000 m/z to 37,000 m/z and 37,500 m/z to 40,000 m/z. In samples from hyperemesis patients abnormal peaks around 32,500 m/z to 34,500 m/z appear, which are indicative of the condition. The peak seen in normal pregnancy samples at 37,089.22 m/z is shifted to a lower m/z, so that abnormal peaks appear at 33,670.68 for H1 and 33,674.34 for H2 in Hyperemesis gravidarum patients as shown in FIG. 3.

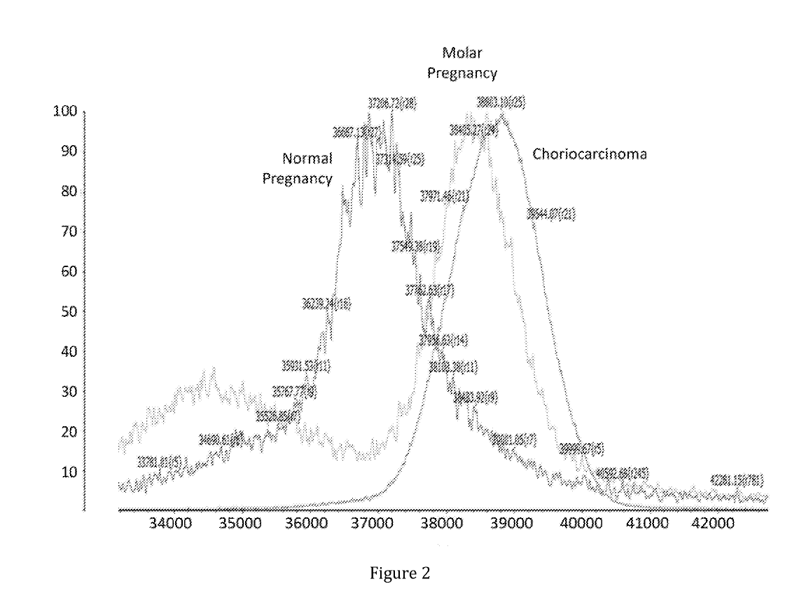

Preferably, the method is used to detect the presence of a gestational trophoblastic disease from analysis of a maternal sample. For gestational trophoblastic diseases (GTD), the preferred range for analysis is 35,000 m/z to 45,000 m/z. A mass shift of the peaks seen within 35,000 m/z to 37,500 m/z in a sample from patients with normal pregnancy occurs in GTD. A shift of the peaks to the range about 37,600 m/z to 39,200 m/z can be indicative of molar pregnancy and, a shift of the peaks to the range about 37,900 m/z to 39,600 m/z can be indicative of choriocarcinoma. In particular a peak around 38,405 m/z is seen in patients with a molar pregnancy and, a peak around 38,803 m/z is seen in patients with choriocarcinoma, as compared to that seen in normal pregnancy, which is around 36,687 m/z, as shown in FIG. 2.

The analysis of the mass spectra can be easily calculated using a suitable computer software program. A computer can also be programmed with the suitable algorithm in order to provide an indication of the likelihood of a pregnancy disorder, or successful implantation.

Direct mass spectral analysis can be carried out by mass spectrometry. Suitable mass spectrometry techniques include fast atom bombardment (FAB), chemical ionization (CI), atmospheric-pressure chemical ionization (APCI), electrospray ionization (ESI), matrix-assisted laser desorption/ionization (MALDI), Quadrupole mass analyzers, ion traps such as quadrupole ion trap, quadrupole ion trap, orbitrap, quadrupole ion trap, Fourier transform ion cyclotron resonance mass spectrometry (FTMS), Ion cyclotron resonance (ICR) or combinations of the above. Preferably, the direct mass spectral analysis can be carried out by MALDI-time of flight (MALDI-TOF) mass spectrometry.

Also described is a method of providing a prognosis for a pregnancy comprising:
a) obtaining a sample
b) subjecting the sample to direct mass spectral analysis; and
c) comparing the spectra resulting from said analysis to mass spectral spectra obtained from a sample from a normal healthy ongoing pregnancy to determine whether said spectra from said sample is indicative of a poor prognosis for pregnancy.

Preferably the method provides a prognosis and/or detection of a disorder of pregnancy, and the spectra resulting from the analysis are compared to those from a woman with a normal healthy pregnancy, in particular a normal healthy singleton pregnancy

Alternatively the method provides a prognosis of successful implantation of a cultured embryo, and the spectra resulting from the analysis are compared to those from the fluid surrounding an embryo which has successfully implanted i.e. resulted in a viable pregnancy.

In this specification, the verb “comprise” has its normal dictionary meaning, to denote non-exclusive inclusion. That is, use of the word “comprise” (or any of its derivatives) to include one feature or more, does not exclude the possibility of also including further features. The word “preferable” (or any of its derivates) indicates one feature or more that is preferred but not essential.

All or any of the features disclosed in this specification (including any accompanying claims, abstract and drawings), and/or all or any of the steps of any method or process so disclosed, may be combined in any combination, except combinations where at least some of such features and/or steps are mutually exclusive.

Each feature disclosed in this specification (including any accompanying claims, abstract and drawings), may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

The application will now be described in the examples below which refer to the following figures:

FIG. 1 illustrates the regions of interest in the spectra generated from a sample.

FIG. 2 shows the MALDI-ToF mass spectrum obtained from the overlay of three spectra from the three samples analyzed in this example; normal, molar and choriocarcinoma pregnancy urine samples. The neat normal pregnancy urine shows a peak at 36,687 m/z represents the normal condition. The molar pregnancy urine, shows a peak at 38,405 m/z within a range of 37,600 m/z to 39,200 m/z and the choriocarcinoma urine sample shows a peak at 38,803 m/z which lies within the 37,900 m/z to 39,600 m/z range.

FIG. 3 shows the MALDI-TOF mass spectra (overlay and then stacked) of expected peak regions in 14,000 m/z to 100,000 m/z range for normal pregnancy urine compared to those of hyperemesis gravidarum. The peaks indicated in the top spectra at 23897 m/z, 36123 m/z and 38405 m/z are indicative of the normal state. In the lower half of the figure peaks at 33670 m/z and 33674 m/z are derived from the urine of women with hyperemesis gravidarum and are shown next to that of a normal pregnancy urine sample at 37089 m/z.

FIG. 4 shows the identification of hot spots in the spectra obtained from samples of culture fluids surrounding embryos which resulted in an ongoing pregnancy, spontaneous abortion, a biochemical pregnancy, negative pregnancy or where the embryo had a chromosomal abnormality.

Looking across the different outcomes regions of variance can be seen. These regions or Hotspots can be compared for example, relatively, as a proportion of each other or as ratios. Regions are identified in terms of Thompson Units (Th) which identify a region of mass:charge (m/z) which is itself proportional to mass in Kda.

Bins can be created any size and in any region where differences can be seen.

FIG. 5 shows separation between ongoing pregnancy and negative pregnancy using three hot spots. Using only three hotspots, 4600-4700 m/z, 6400-6500 m/z, and 9100-9200 m/z a reasonable separation especially between pregnancy and a negative pregnancy can be achieved but there is quite some overlap between pregnancy and pregnancy losses (Biochemical pregnancy and Spontaneous abortion/miscarriage)

FIG. 6 shows a simple correlation comparing subjective visual blastocyst scoring (Gardner) against our Best Embryo Secretome Scoring Test (BESST), the likelihood of an embryo becoming a successful ongoing pregnancy. 1^{st} Click=spearman rank correlation rho=0.4. P=0.005 which is a significant

FIG. 7 shows an example of how hot spots can be identified and selected for us in an algorithm.

FIG. 8 shows the development of an algorithm. By using more complex analysis involving HotSpots and HotSpot regions it is possible to design algorithms which can be built based on outcome, adding in new regions in a stepwise fashion to achieve a minimum of 60% positive predictive values for all outcomes. Arrows indicate increasing probability of identifying blastocysts which will go onto a viable pregnancy to 57%.

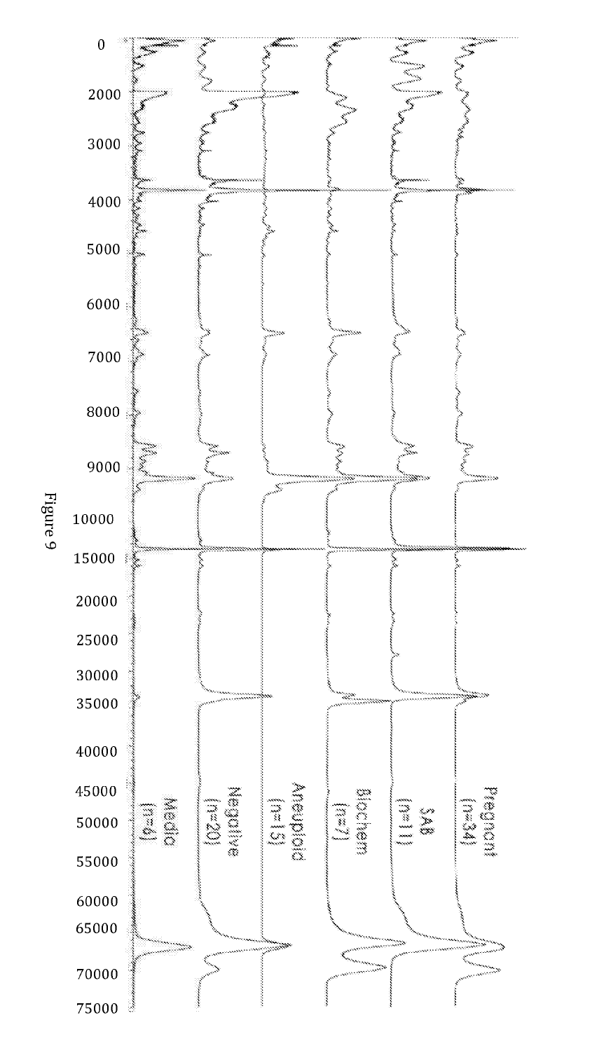

FIG. 9 shows the mass range 2000-80000 m/z for averaged spectra following the MALDI ToF MS of embryo culture fluid. Profiles from ongoing pregnancies are compared with profiles from biochemical pregnancies, spontaneous abortions (SAB), aneuploid embryos and also those which did not implant (negative pregnancies).

FIG. 10 shows Non-normalized averaged spectra for 28 normal controls and 12 preeclampsia samples for 6000-12,000 m/z. Arrows indicate most abundant ion signals where the intensity differs between the pre-eclampsia vs normal controls.

FIG. 11 shows Differential Masses between Preeclampsia and Normal Control Groups Box and whisker plots for selected mass bins that had calculated statistical significance (p≦0.05). m/z=6029 in plot (a), 6538 in plot (b) and 6599 in plot (c).

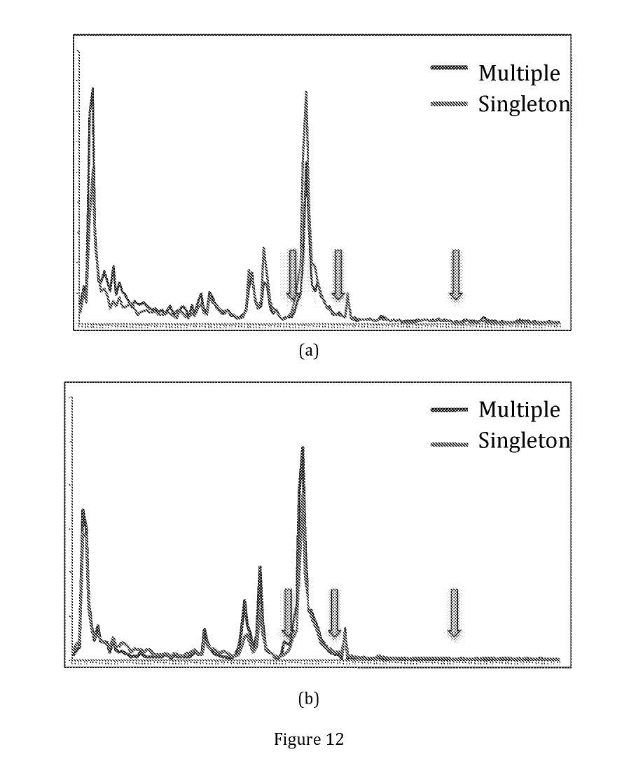

FIG. 12 shows urine spectra (6000-14000 Th) from 5-7 week gestation (a) and 8-11 week gestation (b). Panels compare profiles from multiple embryos with those of singleton embryos for each period in gestation. HotSpots 1, 2 and 3, which were used to build the decision tree discussed below, are indicated by arrows.

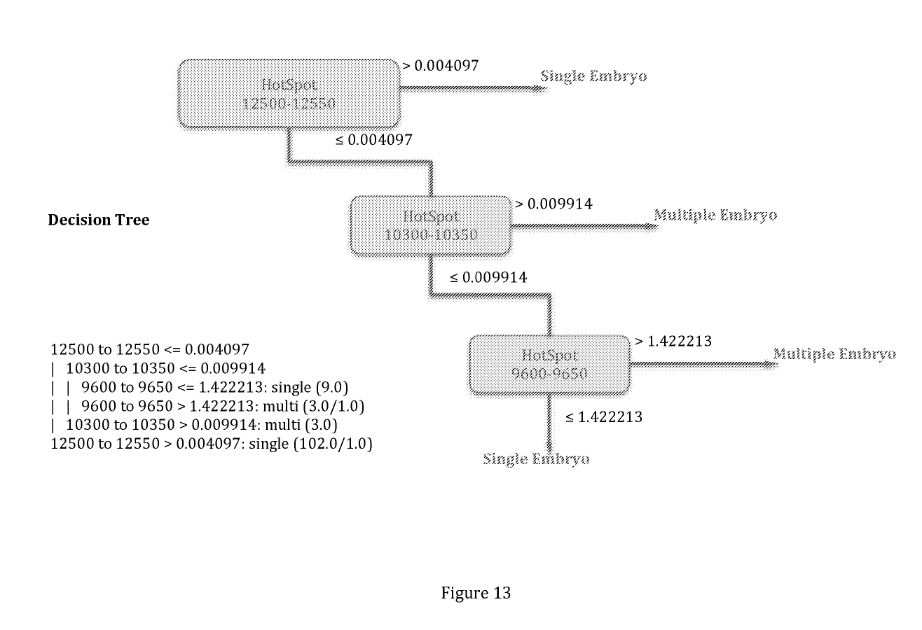

FIG. 13 shows the decision tree used to determine the presence of multiple gestations.

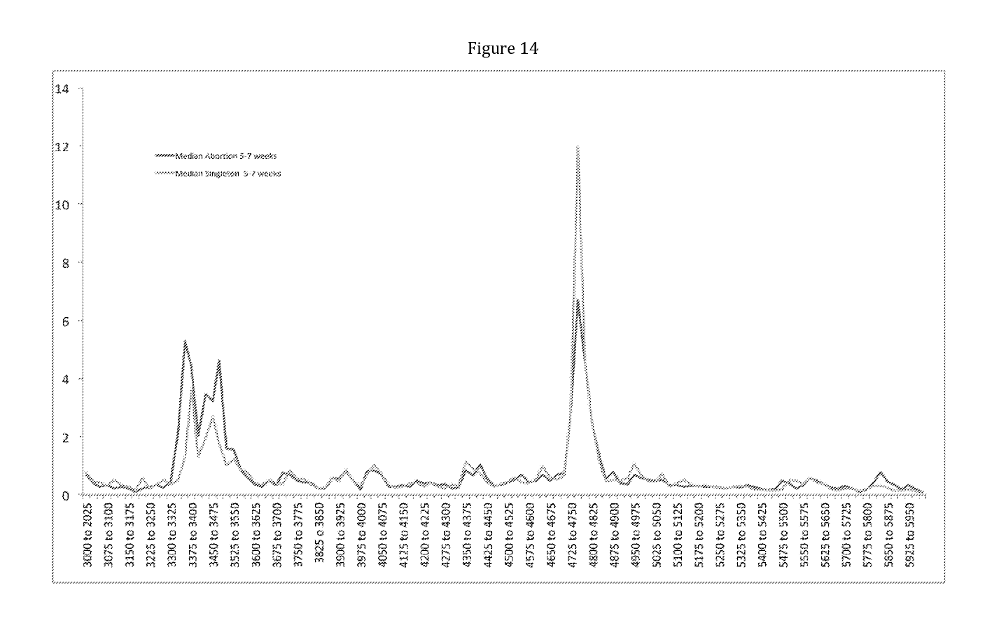

FIG. 14 compares the spectra of samples from women with ongoing pregnancies with those who suffer a spontaneous abortion.

### EXAMPLE 1 GESTATIONAL TROPHOBLASTIC DISEASE

In this example we present a method for the MALDI analysis of m/z variants indicative of the GTD condition.

### Results

Analyses of urine samples from patients with normal pregnancy indicated peaks within the mass region 14,000 m/z to 100,000 m/z. Mass shifts from normal pregnancy with peaks within the range 35,000 m/z to 37,500 m/z to abnormal peaks of 37,600 m/z to 39,200 m/z in molar pregnancy and 37,900 m/z to 39,600 m/z in choriocarcinoma samples. These shifts (in the absence of another peak in a similar range) are indicative of the condition. In this example the abnormal peaks are at 38,405 m/z for molar pregnancy and 38,803 m/z for choriocarcinoma, compared to that seen in normal pregnancy, which was 36,687 m/z in this example. (FIG. 2)

### EXAMPLE 2 HYPEREMESIS GRAVIDARUM

Here we describe a method to identify molecular forms of hCG by discriminating mass shifts in patients with hyperemesis gravidarum.

### Results

Analyses of urine samples from patients with normal pregnancy indicated peaks within the mass region 14,000 m/z to 100,000 m/z. Mass shifts from normal pregnancy with peaks within the range 22,000 m/z to 26,000 m/z, 35,000 m/z to 37,000 m/z and 37,500 m/z to 40,000 m/z to abnormal peaks of 32,500 m/z to 34,500 in samples from hyperemesis patients are indicative of the condition. In this example the abnormal peaks are at 33,670.68 m/z for H1 and 33,674.34 m/z for H2, which are of lower m/z, compared to that seen in normal pregnancy, which was 37,089.22 m/z in this example. (FIG. 3)

### EXAMPLE 3 PRE-ECLAMPSIA

Direct analysis of urine by MALDI-ToF MS reveals changes in protein profile at higher (>greater than 14,000 m/z) and low mass (>2000 m/z-14,000 m/z) ranges in patients developing preeclampsia. (See FIGS. 10 & 11)

These changes do not only characterize clinical pre-eclampsia but changes on the profile of the low mass profiles after conception and during the first trimester may predict those who will develop pre-eclampsia and should receive prophylactic treatments such as aspirin and low molecular weight heparin to reduce maternal and fetal morbidity. Samples obtained from women who went on to develop pre-eclampsia showed changes in the spectra between 6,000-8,000 m/z, In particular changes in the peaks at 6029 m/z, 6538 m/z and/or 6599 m/z (±5 m/z) occurred in patients with pre-eclampsia as shown in FIGS. 10 and 11.

### EXAMPLE 4 EMBRYO/EARLY PREGNANCY

In this example we present a method for the MALDI ToF MS analysis of m/z variants indicative of implantation or implantation potential both in the urine of a woman following embryo transfer, intrauterine insemination or other assisted reproduction method or by natural conception and also the pre-implantation media of embryos in culture for the assessment, diagnosis and prognosis of pregnancy.

### Results

Analyses of urine and embryo culture fluid samples from patients with normal pregnancy indicated peaks within the mass region 2,000 m/z-14,000 m/z and 14,000 m/z to 100,000 m/z. Mass shifts from culture fluid of successfully implanting assisted reproductive technology blastocyst within the range 22,000 m/z to 26,000 m/z, 35,000 m/z to 37,000 m/z and 37,500 m/z to 40,000 m/z to abnormal peaks in 22,000 m/z to 26,000 m/z and 35,000 m/z-40,000 m/z in culture fluid

Urine samples from women post embryo transfer showed distinctive profiles with peaks at 9,700 m/z to 10,000 m/z, comparable to that seen in spontaneous and ongoing conceptions, to either reduced/no peaks in theses region and/or abnormally elevated peaks typically at 7,500 m/z to 8,000 m/z and 10,500 m/z to 12,000 m/z, which was associated with failing implantation and fetal aneuploidy.

In both the analysis of culture fluid and early pregnancy urine it is possible to use variation in mass spectral profiles in specific regions to discriminate vioable ongoing pregnancies from those which will fail (biochemical or SAB) or never result in a positive pregnancy test (negative pregnancy) FIG. 9.

### EXAMPLE 5 PREDICTING PREGNANCY OUTCOMES USING A NON-INVASIVE ANALYSIS OF SECRETOME PARAMETERS IN SPENT BLASTOCYST CULTURE MEDIA USING MALDI-TOF MS

### Study Design, Size, Duration:

A study examining 75 samples of spent media from embryo cultures using MALDI ToF mass spectrometry and subsequent correlation with pregnancy outcomes was carried out.

### Materials, Settings and Methods:

Spent culture media from blastocysts in culture prior to embryo transfer were collected as part of routine ART cycles and stored at −20° C. The samples were shipped frozen to the analytical laboratory and subjected to matrix assisted laser desorption ionization (MALDI), time of flight (ToF) mass spectrometry (MS) as described above.

### Main Results and the Role of Chance:

Data from spectra was collected from the region 12,000 to 50,000 m/z and normalized. Quantitative characteristics of the spectral data were used to compare four groups: Pregnant Ongoing (n=32), Pregnant Spontaneous Abortion (n=11), Pregnant Biochemical (n=9) and Not Pregnant (Negative pregnancy test) (n=23) alongside media controls (n=5). (See FIGS. 4 and 5) This method using MALDI (named Best Embryo Secretome Scoring Test (BESST)), was validated by comparing it with subjective visual blastocyst scoring (Gardner) and the likelihood of an embryo becoming a successful ongoing pregnancy. (FIG. 6)

Algorithms exploiting the m/z variability were designed to predict each outcome and all classifications were assigned using a combination of less than 20 cut-off based criteria. All outcomes could be predicted with 95% accuracy with only 5% incorrectly classified (1fp biochemical, 1fp SAB and 2fp pregnant). (See FIGS. 7 and 8)

### EXAMPLE 6 SPONTANEOUS ABORTION/MISCARRIAGE

A study examining 121 urine samples from women testing positive for pregnancy and attending ART centre in the USA was carried out. Urine from 6-10 weeks gestation was analysed by MALDI ToF MS and subsequently correlated with ongoing singleton pregnancy outcomes. Samples were collected and analyzed between March and December 2014.

### Materials, Settings and Methods:

Urine samples were obtained from women who conceived spontaneously, after IUI, or after ART, and shipped frozen to the analytical laboratory. Once thawed, samples were subjected to matrix assisted laser desorption ionization (MALDI), time of flight (ToF) mass spectrometry (MS) either as neat urine or diluted 10-1000 fold in dH₂O.

### Main Results and the Role of Chance:

Mass spectral data were examined in the region of 6,000 to 14,000 m/z following MALDI ToF MS of urine from pregnant women. Spectral data was normalized and quantitative characteristics of the profile were compared between outcomes: ongoing pregnancy (n=111), and subsequent spontaneous abortion (n=10). Algorithms exploiting the m/z variability were designed to predict outcome with >99% accuracy and only one false negative. Diagnostic decisions were formulated using a decision tree made up of only five m/z based cut-off criteria. (See FIG. 14)

Spectral data was extracted to analysis software as raw mzXML (mass:charge Extensible Markup Language) data to mMass to ASCI (American Standard Code for InformaMon Interchange) files. Data were cropped to 3000-14000 Th (m/z) and were divided into two key regions (Region 1 and Region 2). (See FIG. 14) Normalisation of spectra in Region 2 rendered the profiles comparable and HotSpots at 6000-6050 Th, 6450-6500 Th, 9350-9400 Th, 11150-11200 Th, and 11800-11850 Th were used to make the decision tree shown below.

11150 to 11200<=0.023694: viable (64.0)
11150 to 11200>0.023694

|6000 to 6050<=0.065791

| |9350 to 9400<=0.000015

| | |6450 to 6500<=0.538811: abort (7.0/1.0)

| | |6450 to 6500>0.538811: viable (10.0)

| |9350 to 9400>0.000015: abort (2.0)

|6000 to 6050>0.065791

| |11800 to 11850<=0.185647: viable (36.0)

| |11800 to 11850>0.185647: abort (2.0)

The application of MALDI ToF mass spectrometry is traditionally used to identify target molecules, associated with disease, and then design and develop other means to quantify them. Direct MALDI has been applied successfully to the identification of bacterial in microbiology laboratories to great effect. In this study we also use a direct approach to analyse the spectra of urine in early pregnancy to predict the likelihood of a spontaneous abortion. Our approach disregards the identity of the proteins and peptides and looks instead at changes in profile patterns at certain HotSpots within the spectra. This variability between spectra can be used to create cut-off values. Spectral changes which indicate a future spontaneous abortion, which may lie above or below that cut-off, can be combined to build the algorithms. Therefore, by creating a decision tree involving just a few HotSpots we were able to predict outcomes with >99% accuracy and only one false negative. Variability in averaged urine spectra changes with gestational age and different mathematical approaches can be developed to optimise detection in very early pregnancy (5-7 weeks) when compared to later in the first trimester (8-10 weeks). Further analysis of HotSpots in Region 1 could improve detection algorithms further to better explain early pregnancy losses in women undergoing assisted reproduction.

### EXAMPLE 7 MULTIPLE VS SINGLETON PREGNANCIES

This study represents the first to correlate the spectral analysis of urine by MALDI ToF MS to identify multiple from singleton pregnancies.

### Study Design, Size, Duration:

A study examining 117 urine samples from women testing positive for pregnancy and attending ART centre in the USA was carried out . . . . Urine from 6-10 weeks gestation was analysed by MALDI ToF MS and subsequently correlated with singleton or multiple gestations.

### Materials, Settings and Methods:

Urine samples were obtained from women who conceived spontaneously, after IUI, or after ART, and shipped frozen to the analytical laboratory. Once thawed, samples were subjected to matrix assisted laser desorption ionization (MALDI), time of flight (ToF) mass spectrometry (MS) either as neat urine or diluted 10-1000 fold in dH₂O.

Main Results and the role of chance:

Mass spectral data were examined in the region of 6,000 to 14,000 m/z following MALDI ToF MS of urine from pregnant women. Spectral data was normalized and quantitative characteristics of the profile were compared between outcomes: ongoing singleton pregnancy (n=111) ongoing twin or triplet pregnancy (n=6). Algorithms exploiting the m/z variability were designed to predict outcome with >98% accuracy. Diagnostic decisions were formulated using a decision tree made up of only three m/z based cut-off criteria. (See FIGS. 12 and 13)

Unlike traditional immunoassay based and ultrasound approaches, MALDI ToF MS of very early maternal urine represents an accurate, rapid, and non-invasive method of determining pregnancy outcome and success in women who are trying to conceive. Rapid and non invasive early diagnosis of multiple gestations represents a valuable new tool in the management of women undergoing ART and spontaneous pregnancy.

### EXAMPLE 8 NON-INVASIVE ANALYSIS OF SECRETOME PARAMETERS IN SPENT BLASTOCYST CULTURE MEDIA USING MALDI-TOF MS TO IDENTIFY ANEUPLOIDY: POTENTIAL ALTERNATIVE TO PREIMPLANTATION GENETIC SCREENING

### Objective:

To identify aneuploid marker parameters in mass spectral profiles of spent media from blastocyst culture by MALDI ToF MS.

### Design:

Prospective pilot study examining spent media from blastocyst cultures using MALDI ToF mass spectrometry to identify pattern differences in aneuploid blastocysts. Media samples and controls were collected, shipped, stored and analyzed between December 2014 and April 2015.

### Materials and Methods:

Spent culture media from blastocysts in culture prior to embryo transfer were collected as part of 40 routine ART cycles and stored at −20° C. Blastocyst grading was carried out according to Gardner's criteria. The media samples were shipped frozen to the analytical laboratory and subjected to matrix assisted laser desorption ionization (MALDI), time of flight (ToF) mass spectrometry (MS). Data from spectra were collected from the region 3,000 to 30,000 m/z, smoothed and normalized. Qualitative characteristics of the data from spectral hotspots in 6000 m/z to 9500 m/z regions were examined for difference.

### Results:

After blastocyst grading 19 were assigned 5AA grades and correlated well with positive pregnancy ( 16/19, 84.2%), 18 were given mixed grades but resulted in a negative pregnancy, 3 had an abnormal PGS and were not graded or transferred. Secretome patterns following MALDI ToF MS analysis in the 6000 m/z to 9500 m/z regions show distinct pattern differences between high grade embryos, embryos which resulted in a negative pregnancy, and aneuploid embryos. In particular, aneuploid blastocysts give rise to significant profile differences in the 8500-9000 m/z regions such that they are completely distinct from either high grade embryos or embryos which resulted in a negative pregnancy.

### Conclusion:

Non-invasive analysis of spent blastocyst culture media by MALDI ToF MS can identify aneuploid blastocysts amongst other high and low quality blastocysts.

### EXAMPLE 9 NON-INVASIVE ANALYSIS OF SECRETOME PARAMETERS IN SPENT BLASTOCYST CULTURE MEDIA USING MALDI-TOF MS CORRELATES WITH BLASTOCYST GRADING AND PREGNANCY OUTCOME

### Objective:

To investigate correlations between non-invasive proteomic analysis of spent blastocyst culture media by MALDI ToF MS and traditional blastocyst grading.

### Design:

Prospective pilot study examining 90 samples of spent media from embryo cultures using MALDI ToF mass spectrometry and subsequent correlation with blastocyst grading scores. Samples were collected, shipped, stored and analyzed for one year between April 2014 and April 2015.

### Materials and Methods:

Spent culture media from blastocysts in culture prior to embryo transfer were collected as part of 90 routine ART cycles and stored at −20° C. Blastocyst grading was carried out using Gardner's criteria. Following blastocyst culture 3 samples were excluded based on abnormal PGS results (N=3), and 1 FET case was excluded in the absence of grade. 86 samples were graded 5AA to 3CC and 6 early blastocysts were graded 1BB. Grades were assigned a numerical value (1 to 11) and ranked. The media samples were shipped frozen to the analytical laboratory and subjected to matrix assisted laser desorption ionization (MALDI), time of flight (ToF) mass spectrometry (MS). Data from spectra were collected from the region 3,000 to 30,000 m/z, smoothed and normalized. Quantitative characteristics of the data from spectral hotspots were used to rank blastocysts. Using Stats Direct software ranked blastocyst scores were correlated with traditional blastocyst grading by spearman rank correlation. Rank scores obtained from MALDI ToF MS analysis was also correlated with pregnancy outcome.

### Results:

There was a good significant correlation (Rho 0.33, P=0.02) between traditional grading of blastocysts and secretome analysis scoring following MALDI ToF MS. Quantitative characteristics of the spectral data were also used to compare pregnancy outcome with spectral scoring in two groups (pregnant and negative pregnancy—biochemical pregnancies and spontaneous abortions were excluded). Algorithms exploiting the m/z variability were designed to predict each outcome and all classifications were assigned using a combination of less than 20 cut-off based criteria.

### Conclusion:

Non-invasive analysis of spent blastocyst culture by MALDI ToF MS correlates with traditional methods of blastocyst grading and pregnancy outcome.

1. A method of providing a prognosis for a pregnancy, comprising subjecting a sample to direct mass spectral analysis.

2. The method of claim 1 for providing a prognosis of a successful ongoing pregnancy.

3. The method of claim 1 for providing a prognosis of successful implantation of a cultured embryo.

4. The method of claim 3, wherein said method can differentiate between a successful implantation leading to an ongoing pregnancy or a negative pregnancy.

5. The method of claim 1 wherein the sample comprises fluid surrounding an embryo in culture.

6. The method of claim 5, wherein a chromosomal abnormality is detected in the embryo.

7. The method of claim 6, wherein said chromosomal abnormality is an aneuploidy.

8. The method of claim 1 for providing a prognosis of a disorder of pregnancy.

9. The method of claim 8 wherein said disorder of pregnancy is selected from pre-eclampsia, Hyperemesis Gravidarum, or Gestational trophoblastic diseases.

10. The method of claim 8 wherein said disorder of pregnancy is a spontaneous abortion or a multiple pregnancy.

11. The method of claim 1 wherein the sample is obtained from a pregnant woman.

12. The method of claim 11 wherein the sample is selected from urine, blood or serum sample.

13. The method of claim 1 wherein the sample is diluted prior to direct mass spectral analysis.

14. A method according to claim 1, wherein the spectra obtained from the direct mass spectral analysis is normalized.

15. A method according to claim 1, wherein each sample is compared against a reference spectral model of expected mass between about 500 m/z-100,000 m/z determined from statistical analysis of a collection of samples obtained from normal ongoing pregnancies.

16. A method according to claim 1, wherein the mass spectral analysis carried out by mass spectrometry.

17. A method according to claim 11, wherein the mass spectrometry is matrix-assisted laser desorption/ionization time of-flight mass spectrometry (MALDI-ToF MS).
